(19) United States
(12) Patent Application Publication
(10) Pub. No.: US 2023/0082598 A1
(43) Pub. Date: Mar. 16, 2023

(54) DIAGNOSTIC APPARATUS FOR CHRONIC OBSTRUCTIVE PULMONARY DISEASE BASED ON PRIOR KNOWLEDGE CT SUBREGION RADIOMICS

(71) Applicant: ZHEJIANG LAB, Hangzhou (CN)

(72) Inventors: Wentao ZHU, Hangzhou (CN); Hui SHEN, Hangzhou (CN); Ling CHEN, Hangzhou (CN); Yuan JIN, Hangzhou (CN); Hailiang HUANG, Hangzhou (CN)

(21) Appl. No.: 17/740,349

(22) Filed: May 10, 2022

(30) Foreign Application Priority Data
Sep. 10, 2021 (CN) 202111061904.1

(51) Int. Cl. G06T 7/00; A61B 6/00; G06T 7/11; G06V 10/44; G06V 10/764

(52) U.S. Cl. CPC G06T 7/0012; A61B 6/5205; G06T 7/11; G06V 10/457; G06V 10/764; G06T 2207/10081; G06T 2207/30061; G06V 2201/02

(57) ABSTRACT

Disclosed is a diagnostic apparatus for a chronic obstructive pulmonary disease (COPD) based on prior knowledge CT subregion radiomics, belonging to the field of medical imaging. The diagnostic apparatus comprises: a subregion partitioning module based on prior knowledge configured for partitioning a CT lung image of a patient into three subregions based on the CT values of the interior of the lung, wherein the CT value of the interior of the lung of a subregion 1 is in the range of (−1024, −950), the CT value of the interior of the lung of a subregion 2 is in the range of (−190, 110), and the CT value of the interior of the lung of a subregion 3 is in the range of (−950, −190); a feature extraction module configured for extracting the radiomics features of the three subregions, respectively, and obtaining the LAA-950I features.

### CROSS-REFERENCE TO RELATED APPLICATIONS

The present application claims priority to Chinese Patent Application No. 202111061904.1, filed on Sep. 10, 2021, the content of which is incorporated herein by reference in its entirety.

### TECHNICAL FIELD

The present disclosure relates to the field of medical imaging, and more particularly to a diagnostic apparatus for a chronic obstructive pulmonary disease based on prior knowledge CT subregion radiomics.

### BACKGROUND

A chronic obstructive pulmonary disease (COPD), characterized by irreversibleairflow limitation, is one of the main causes of death, and the risk of a primary lung cancer in patients with COPDs is also increased. More than 65 million people worldwide are affected by COPDs. Early diagnosis of chronic obstructive pneumonia has been shown to have a positive effect on slowing down its clinical progression and improving the quality of life of patients. However, in fact, missed diagnosis and misdiagnosis of COPDs are very common (about 60%-86%), which means that many COPDs may miss the opportunity of optimal preventive and therapeutic management to slow the clinical progress. Therefore, the early diagnosis of the chronic obstructive pneumonia is an urgent problem to be solved.

Pulmonary function tests have been widely used to identify and discriminate the severity of pulmonary airflow obstruction and are the gold standard for COPDs. However, a slight negligence in lung function tests can lead to large test deviations. Quantitative CT has proved to be an important method for evaluating the chronic obstructive pneumonia, which can reduce the rate of misdiagnosis, thereby preventing disease progression, complications, and improving management and early mortality. A series of studies have shown that CT radiomics plays a positive role in the diagnosis of the chronic obstructive pneumonia, improving clinical treatment capabilities, and providing decision-makings. However, the existing CT radiomics feature extraction is to analyze and evaluate the entire lung as a whole. In fact, the structure in the lung is also complex, including different parts such as small bronchi, air in alveoli, lung tissue, etc., and small bronchial obstruction and alveolar air retention are important criteria for the diagnosis of COPDs. Therefore, CT subregion radiomics can achieve more careful observation of different structures in the lung and extract the features of different structures separately, which has a more positive effect on improving the diagnosis efficiency of COPDs.

### SUMMARY

In view of the deficiencies of the prior art and taking account of the various structures in the lung, it is an object of the present disclosure to provide a diagnostic apparatus for chronic obstructive pneumonia based on the radiomics features of CT image subregions.

The technical solution employed by the present disclosure is as follows:

a diagnostic apparatus for a chronic obstructive pulmonary disease based on prior knowledge CT subregion radiomics, comprising:

a subregion partitioning module configured to partition a CT lung image of a patient into three subregions based on the CT values of an interior of a lung, the CT value of the interior of the lung in a subregion 1 is in a range of (−1024, −950), the CT value of the interior of the lung in a subregion 2 is in a range of (−190, 110), and the CT value of the interior of the lung in a subregion 3 is in a range of (−950, −190);

a feature extraction module configured to extract radiomics features of the three subregions, respectively, and to obtain LAA-950I features;

a classification module configured to distinguish whether the patient has a chronic obstructive pulmonary disease based on the radiomics features of the three subregions and the LAA-950I features extracted by the feature extraction module.

Furthermore, the radiomics features are in particular shape features, texture features and/or statistical features.

Furthermore, the classification module adopts a support vector machine (SVM) classification model, a decision tree classification model, or a logistic regression classification model.

Furthermore, the feature extraction module is further configured to extract a connected domain feature of the subregion 1, the connected domain feature being a percentage of a connected domain volume in the subregion 1 to an entire lung volume in an image.

Furthermore, the connected domain feature includes three connected domain features corresponding to the first three connected domains in the subregion 1 in terms of volume from the greatest to the smallest.

The diagnostic process of the apparatus of the present disclosure is:

CT subregion partitioning based on a priori knowledge: a CT lung image of a patient is partitioned into three subregions.

Feature extraction: radiomics features are extracted for each CT subregion, and new features are designed and extracted based on medical knowledge, including LAA-950I features and connected domain features.

It is judged whether it is a chronic obstructive pulmonary disease according to the extracted features.

The beneficial effect of the present disclosure is that the apparatus of the present disclosure partitions the lung into different subregions by threshold segmentation based on medical prior knowledge, focusing on the evaluation of different regions, such as alveolar air entrapment regions, lung tissue or fine bronchial obstruction, and the like. In medicine, low attenuation areas of the lung (areas below −950 HU in CT) play an important role in suggesting emphysema. In contrast, LAA-950, the percentage of the low attenuation area in the entire lung volume, is usually used as an emphysema index. The area with a CT threshold between (−190, 110) generally represents glands and soft tissues and can be suggestive of bronchi in that lung. Therefore, the apparatus of the invention extracts the features of different structures respectively by partitioning a lung into subregions, thereby playing a more positive role in improving the diagnosis efficiency of the chronic obstructive pulmonary disease.

### BRIEF DESCRIPTION OF DRAWINGS

FIG. 1 is a block diagram of the apparatus of the present disclosure;

FIG. 2 is a flow chart of the diagnosis of the apparatus of the present disclosure.

### DESCRIPTION OF EMBODIMENTS

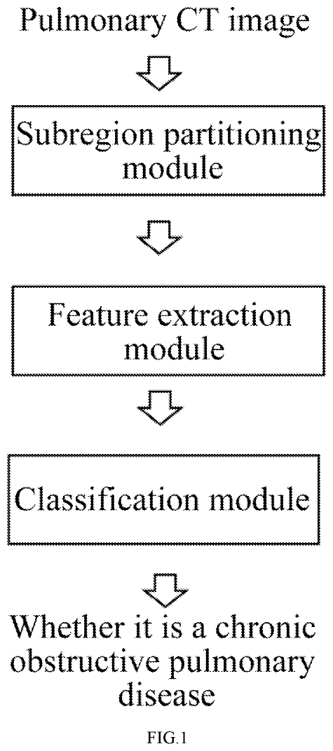

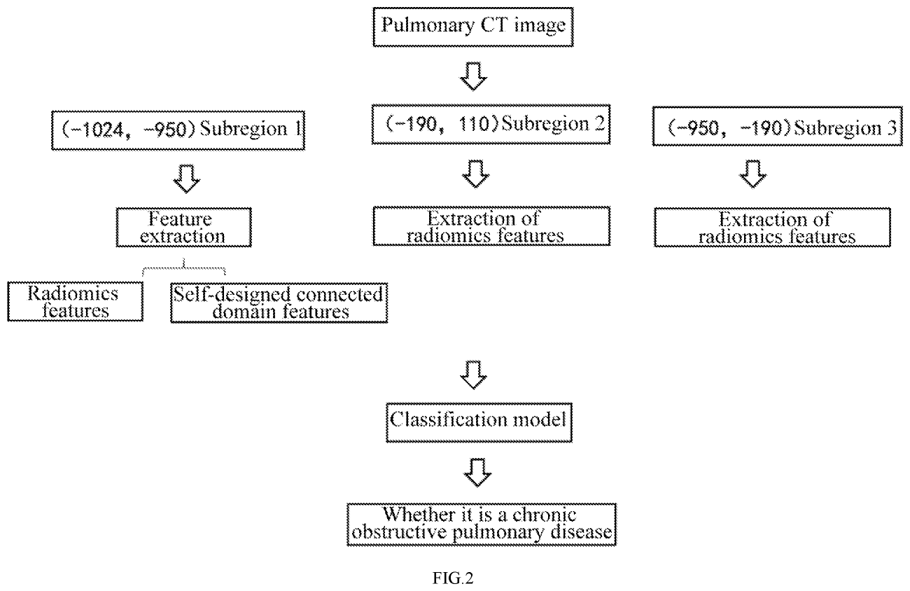

In the present disclosure, the lung is partitioned into different subregions based on a priori knowledge, and then radiomics feature extraction is performed (FIG. 2), so as to better focus on the structures in the lung, such as alveolar air retention, fine bronchi and other structures, thereby improving the diagnostic effect of the chronic obstructive pneumonia. The structure of the diagnostic apparatus is shown in FIG. 1, including:

A subregion partitioning module configured to partition a CT lung image of a patient into three subregions according to a priori knowledge, which is specifically as follows:

(1) The part of the lung with a CT value between (−1024, −950) is partitioned and the partitioned part is a subregion 1, which indicates an air value and can indicate the relevant situation of emphysema;

(2) The part of the lung with a CT value between (−190, 110) is partitioned and the partitioned part is a subregion 2, which indicates glands and soft tissues, and can indicate the situation of the bronchus in the lung;

(3) The part of the lung with a CT value between (−950, −190) is partitioned and the partitioned part is a subregion 3, which indicated the lung condition except the alveoli and bronchi.

A feature extraction module is used for extracting the radiomics features of the three subregions, respectively, and obtaining the LAA-950I features.

LAA-950I is characterized by the percentage of the volume less than 950HU in the whole lung volume, and the specific calculation formula is as follows:

$$LAA - {950}_{I} = \frac{Vol ( < 950 HU )}{Vol ( Lung )} \times 100 \%$$

The radiomics features include shape features, texture features, statistical features, and the like, which are extracted based on pyradiomics tools in this embodiment.

A classification module is used for distinguishing whether a patient has a COPD based on the extracted radiomics features of the three subregions and the LAA-950I features.

Among them, the classification module can use SVM, decision tree, logistic regression and other classification models. The classification module needs to be trained in advance by using the CT lung images with existing diagnostic labels input into the subregion partitioning module and the feature extraction module.

In an embodiment, the feature extraction module further extracts three connected domain features in the subregion 1. Usually the low attenuation areas of the lung (areas with a CT value less than −950) indicate emphysema. The calculation of the size of the emphysema area can assess the severity of abnormal continuous expansion of the air cavity of the respiratory bronchus of the lung. Therefore, the severity of emphysema can be assessed to a certain extent by calculating the size of the first three connecting domains of the subregion 1 from the greatest to the smallest. Therefore, the present disclosure also extracts three connected domain features in the subregion 1.

Among them, the connected domain feature is the percentage of the volume of the first three connecting domains of the subregion 1 from the greatest to the smallest to the entire lung volume, and the specific acquisition process is:

1) converting the image of the subregion 1 obtained by the partitioning into a binary image;

2) using OpenCV to obtain all connected domain information;

3) sorting all connected domains according to their volume from the greatest to the smallest, and obtaining the volumes of Connected_Vol_{No.1}, Connected_Vol_{No.2}, Connected_Vol_{No.3} of the first to third connected domains;

4) calculating the percentages of the volumes of the first to third connected domains to the entire lung volume, Connected_feature_{No.1}m Connected_feature_{No.2}, Connected_feature_{No.3}, as the three connected domain features in the subregion 1, and the calculation formula is as follows:

$${Connected\_feature}_{No . 1} = \frac{{Connected\_Vol}_{No . 1}}{Vol ( Lung )} \times 100 \%$$

$${Connected\_feature}_{No . 2} = \frac{{Connected\_Vol}_{No . 2}}{Vol ( Lung )} \times 100 \%$$

$${Connected\_feature}_{No . 3} = \frac{{Connected\_Vol}_{No . 3}}{Vol ( Lung )} \times 100 \%$$

where Vol (Lung) is the lung volume.

It is to be understood that the above-described embodiments are merely illustrative for clarity of illustration and are not intended to define the embodiments. Other variations or changes may be made by one of ordinary skill in the art in light of the above description. All embodiments need not and cannot be listed exhaustively here. Obvious variations or changes thus extended shall still fall within the scope of the present disclosure.

What is claimed is:

1. A diagnostic apparatus for a chronic obstructive pulmonary disease based on prior knowledge CT subregion radiomics, comprising:
a subregion partitioning module configured to partition a CT lung image of a patient into three subregions based on the CT values of an interior of a lung, wherein the CT value of the interior of the lung in a subregion 1 is in a range of (−1024, −950), the CT value of the interior of the lung in a subregion 2 is in a range of (−190, 110), and the CT value of the interior of the lung in a subregion 3 is in a range of (−950, −190);
a feature extraction module configured to extract radiomics features of the three subregions, respectively, and to obtain LAA-950I features; wherein the feature extraction module is further configured to extract a connected domain feature of the subregion 1, the connected domain feature being a percentage of a connected domain volume in the subregion 1 to an entire lung volume in an image; the connected domain feature comprises three connected domain features corresponding to the first three connected domains in the subregion 1 in terms of volume from the greatest to the smallest;
a classification module configured to distinguish whether the patient has a chronic obstructive pulmonary disease based on the radiomics features of the three subregions and the LAA-950I features extracted by the feature extraction module.

2. The diagnostic apparatus for a chronic obstructive pulmonary disease according to claim 1, wherein the radiomics features are in particular shape features, texture features and/or statistical features.

3. The diagnostic apparatus for a chronic obstructive pulmonary disease according to claim 1, wherein the classification module adopts a support vector machine classification model, a decision tree classification model, or a logistic regression classification model.
